(19) United States
(12) Patent Application Publication
(10) Pub. No.: US 2002/0188210 A1
(43) Pub. Date: Dec. 12, 2002

(54) Pulse wave sensor and pulse rate detector

(76) Inventor: Nobuyuki Aizawa, Gunma (JP)

Correspondence Address: KANESAKA & TAKEUCHI, 1423 Powhatan Street, Alexandria, VA, 22314, US

(21) Appl. No.: 10/152,818

(22) Filed: May 23, 2002

(30) Foreign Application Priority Data
Jun. 11, 2001 (JP) 2001-175909

(51) Int. Cl. A61B 5/02

(52) U.S. Cl. 600/503; 600/502

(57) ABSTRACT

A pulse wave sensor for detecting a pulse wave by detecting light output from a light emitting diode and reflected from the artery of a wrist of a subject, the sensor comprising four photodetectors disposed around the light emitting diode symmetrically on a circle concentric to the light emitting diode, and a pulse rate detector comprising the pulse wave sensor and means of computing the pulse rate of a subject based on the output of the pulse wave sensor.

### BACKGROUND OF THE INVENTION

1. Field of the Invention

The present invention relates to a pulse wave sensor for detecting the pulse wave of a subject from light reflected from a red corpuscle in the artery of a wrist of the subject by irradiating the artery of the wrist with light having a wavelength of an infrared range and to a pulse rate detector for detecting the pulse rate of the subject from the above pulse wave data.

2. Description of the Prior Art

In recent years, along with shift to the aging society and westernized eating habits, an increase in the number of diseases caused by life habits, such as hyperpiesia, diabetes mellitus, heart diseases and cerebrovascular diseases of the brain is becoming a big social problem. As means of preventing these diseases or treating the diseases, a personal exercise cure such as walking is widely adopted. In this exercise cure, a pedometer or kinetic calorimeter is carried to know the quantity of motion. There has recently been proposed a method of estimating a burden on the heart of a person who takes exercise by real-time measuring his/her heart rate at the time of exercise.

For the measurement of the above heart rate, an optical pulse wave sensor for detecting the pulse wave of a subject from reflected light or transmitted light by irradiating the site of a blood vessel with light having an infrared or near infrared range is widely used. Stated more specifically, a pulse wave sensor which comprises a pair of an LED (light emitting diode) and a phototransistor (photodetector) is attached to a finger or ear to measure the heart rate by calculating the cycle (frequency) of pulse waves from the waveform of reflected light or transmitted light detected by the above photodetector.

However, although the conventional pulse wave sensor to be attached to the finger or ear is small in size, a signal from the sensor is weak because it detects the motion of a red corpuscle in the capillary and is easily affected by noise caused by the shaking of the body of the subject. Also, as some pressure is applied to the measurement site at the time of detection, the subject cannot carry the detector for a long time when walking or the like.

Meanwhile, since a strong signal is obtained when the motion of a red corpuscle in the artery is detected, a detector to be attached to a wrist or arm is conceivable. As understood when the pulse of the wrist is actually taken, it is difficult to attach the sensor to a predetermined position. When the attachment position is dislocated, no output can be obtained, thereby making it difficult to implement the detector.

### SUMMARY OF THE INVENTION

It is an object of the present invention which has been made in view of the above problem to provide a pulse wave sensor which is easily attached and is capable of detecting a pulse wave accurately and a pulse rate detector comprising this pulse wave sensor.

According to a first aspect of the present invention, there is provided a pulse wave sensor for detecting a pulse wave by detecting light output from a light emitting diode and reflected from the artery of a wrist of a subject, the sensor comprising at least three photodetectors disposed around the light emitting diode and not linearly. Even when the attachment position of the sensor is dislocated, a pulse wave can be detected accurately.

According to a second aspect of the present invention, there is provided a pulse sensor, wherein a near infrared LED which is a general-purpose product is used as the light emitting diode. This makes it possible to produce an inexpensive sensor.

According to a third aspect of the present invention, there is provided a pulse sensor, wherein the photodetectors are disposed at an equal distance from the light emitting diode.

According to a fourth aspect of the present invention, there is provided a pulse sensor, wherein cavities are formed in a contact face between a holder for holding the light emitting diode and the photodetectors and the wrist, the light emitting face of the light emitting diode and the light receiving faces of the photodetectors are disposed at respective predetermined distances from the contact face, and the sectional forms of the cavities are tapered such that their widths increase toward the contact face. Since this makes it possible to expand the light emitting area and the light receiving area, a pulse wave can be easily detected even when the attachment position of the sensor is dislocated.

According to a fifth aspect of the present invention, there is provided a pulse sensor, wherein a transparent plate-like member is provided on a portion including at least the light emitting face and the light receiving faces of the contact face. This makes it possible to improve adhesion between the sensor and the wrist and thereby further improve the detection efficiency of pulse waves.

According to a sixth aspect of the present invention, there is provided a pulse rate detector comprising the pulse wave sensor of claim 1 and means of computing the pulse rate of a subject based on the output of the pulse wave sensor.

According to a seventh aspect of the present invention, there is provided a pulse rate detector which comprises a transmitter for transmitting the measured pulse rate data to a display for displaying the pulse rate data and a device for computing the amount of motion load from the pulse rate.

The above and other objects, advantages and features of the present invention will become apparent from the following description when taken in conjunction with the accompanying drawings.

### BRIEF DESCRIPTION OF THE DRAWINGS

FIGS. 1 are schematic diagrams of a pulse rate detector according to an embodiment of the present invention;

FIG. 2 is a diagram showing that the pulse rate detector is attached.

FIG. 3 is a schematic diagram of a pulse wave which is the output of a photodetector;

FIGS. 4 are diagrams showing other arrangements of a light emitting diode and photodetectors according to the present invention; and

FIG. 5 is a diagram showing a pulse rate detector according to another embodiment of the present invention.

### DETAILED DESCRIPTION OF THE PREFERRED EMBODIMENTS

Preferred embodiments of the present invention will be described hereinbelow with reference to the accompanying drawings.

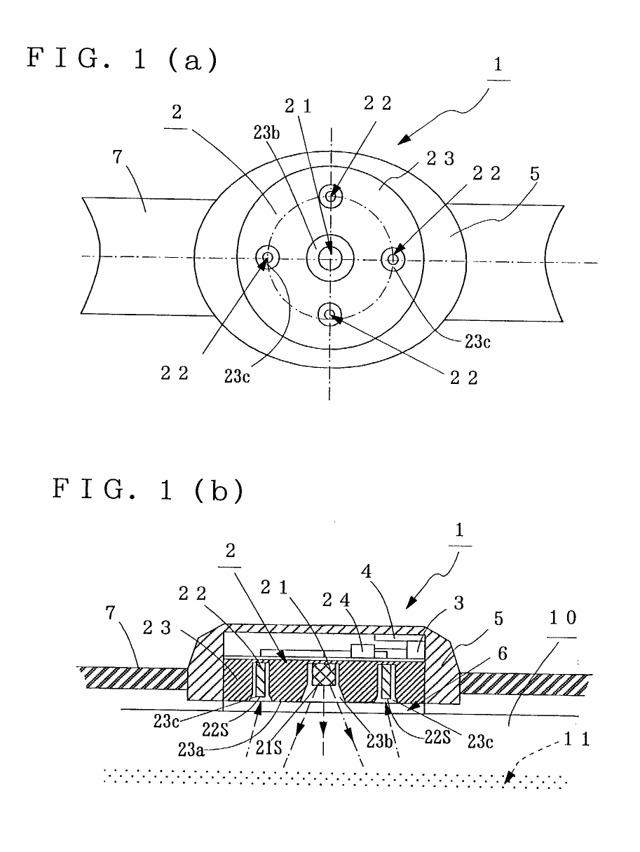

FIGS. 1(a) and 1(b) are schematic diagrams of a pulse rate detector according to an embodiment of the present invention. FIG. 1(a) is a plan view and FIG. 1(b) is a sectional view of the pulse rate detector when it is attached. In these figures, reference numeral 2 denotes a pulse wave sensor which comprises an LED 21 (to be referred to as “light emitting diode” hereinafter) for emitting light having a wavelength of a near infrared range, four phototransistors 22 (to be referred to as “photodetectors” hereinafter) disposed around the light emitting diode 21 symmetrically on a circle concentric to the light emitting diode 21, a holder 23 for storing the above light emitting diode 21 and the photodetectors 22, and a drive detection circuit 24 for detecting a pulse wave by amplifying the outputs of the photodetectors 22, 3 is an arithmetic circuit for computing a pulse rate from the detected pulse wave data, 4 a transmitter for transmitting the above pulse rate data to an unshown display, 5 an outer casing for storing the above pulse wave sensor 2, the arithmetic circuit 3 and the transmitter 4, 6 an acrylic transparent plate mounted to the detection face 23a of the holder 23 to be described hereinafter, and 7 an attachment belt attached to the above outer casing.

The above light emitting diode 21 and the above photodetectors 22 are stored in cavities 23b and 23c formed in the detection face 23a which is a contact side between the holder 23 and a wrist 10, respectively, at positions where the light emitting face 21s of the light emitting diode 21 and the light receiving faces 22s of the photodetectors 22 are set back from the above detection face 23a. In this embodiment, to expand the light emitting area of the light emitting diode 21 and the light receiving areas of the photodetectors 22, the sectional forms of the above cavities 23b and 23c are tapered such that their widths increase toward the contact face.

A description is subsequently given of the method of measuring a pulse rate.

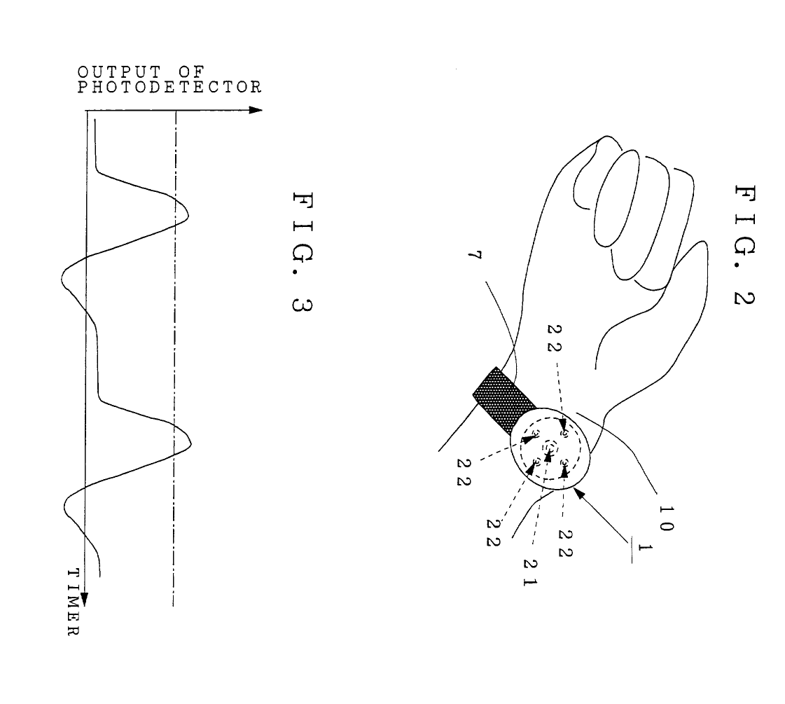

As shown in FIG. 2, a subject carries the above pulse rate detector 1 on the inner side of his/her wrist 10 with a belt in such a manner that the light emitting face 21s of the light emitting diode 21 faces down (on the wrist 10 side). As shown in FIG. 1(b), the above belt 7 is fastened such that the acrylic transparent plate 6 becomes close to the artery 11 of the wrist 10. Thereby, adhesion between the wrist 10 and the pulse rate detector 1 is improved. When the pulse rate detector 1 is attached to the wrist 10 with the belt 7, pulse wave data can be detected at the same pressure as that for attaching a wrist watch with a belt. Therefore, the wrist 10 is not pressed hard, thereby making it possible to carry it for a long time.

Near infrared radiation output toward the wrist 10 from the light emitting diode 21 is reflected by a red corpuscle running through the artery 11 of the wrist 10 and this reflected light is detected by the plurality of photodetectors 22 so as to detect a pulse wave (see FIG. 1(b)). Since four photodetectors 22 are disposed around the light emitting diode 21 on a circle concentric to the light emitting diode 21 in this embodiment, even when the attachment position of the pulse rate detector 1 is dislocated, one of the photodetectors 22 is located near the artery 11, thereby making it possible to detect a pulse wave accurately. If the plurality of photodetectors 22 are disposed linearly, all of the photodetectors 22 may be far from the artery 11. Therefore, it is desired that the photodetectors 22 should not be disposed linearly.

FIG. 3 schematically shows the waveform of a pulse wave which is the output of the above photodetector 22. The detected pulse wave data is amplified by the drive detection circuit 24 and the amplified pulse wave data is transmitted to the arithmetic circuit 3. The arithmetic circuit 3 has a threshold value and computes the number of outputs above the threshold value per unit time so as to calculate a pulse rate and the transmitter4 transmits the pulse rate to a display for displaying the above pulse rate data and a device for computing the amount of motion load. Since the output of the above photodetector 22 is generally low, after the output is amplified, the amplified output is converted into a digital signal for the computation of a pulse rate in this embodiment.

According to this embodiment, the pulse wave of the wrist 10 of the subject is detected by the pulse wave sensor 2 which comprises the light emitting diode 21 for emitting light having a wavelength of a near infrared range and four photodetectors 22 disposed around the light emitting diode 21 symmetrically on a circle concentric to the light emitting diode 21, and a pulse rate is computed from the pulse wave data by the arithmetic circuit 3. Therefore, even when the attachment position of the pulse rate detector 1 is dislocated, a pulse wave can be detected accurately.

Since the acrylic transparent plate 6 is provided on the detection face 23a of the holder 23, adhesion between the pulse rate detector 1 and the wrist 10 can be improved, thereby further improving the detection efficiency of a pulse wave.

In this embodiment, the pulse rate detector 1 is attached with the same pressure as that for attaching a timepiece to the wrist with a belt. Therefore, the subject can carry the pulse rate detector 1 for a long time without pressing his/her wrist excessively.

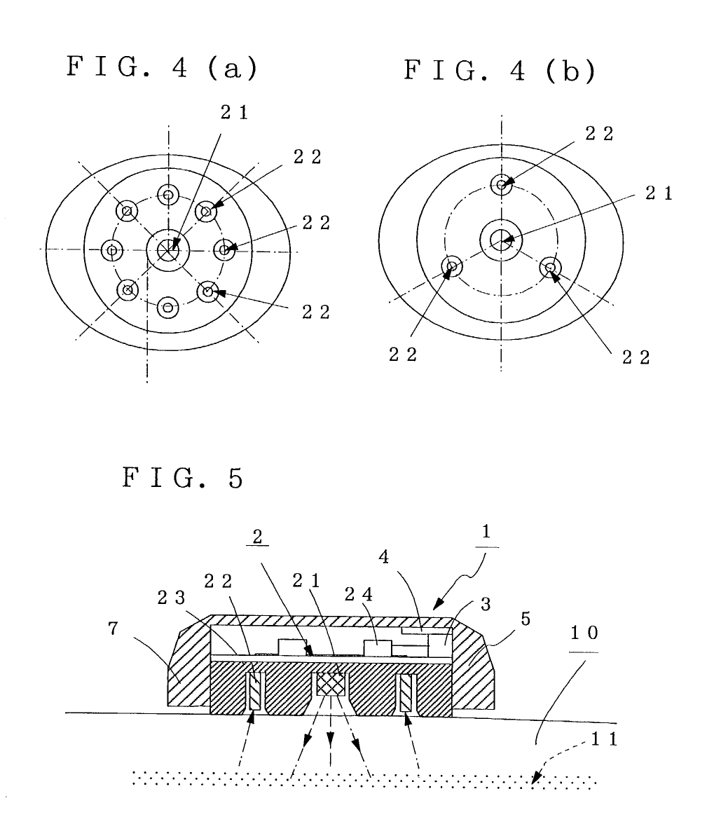

In the above embodiment, four photodetectors which are disposed symmetrically are used to detect the pulse wave of the wrist 10. The arrangement of the light emitting diode 21 and the photodetectors 22 is not limited to this. For example, to further improve detection efficiency, as shown in FIG. 4(a), the number of the photodetectors 22 may be increased. Alternatively, to reduce the size of the pulse rate detector 1, as shown in FIG. 4(b), the number of photodetectors may be reduced. In either case, it is desired that the photodetectors 22 should be disposed around the light emitting diode 21 on a circle concentric to the light emitting diode 21 to detect a pulse wave accurately even when the attachment position of the pulse rate detector 1 is dislocated.

In the above embodiment, a plurality of photodetectors 22 are provided for one light emitting diode 21. The same effect can be obtained when the number of photodetectors 22 is 1 and a plurality of light emitting diodes 21 are disposed around the photodetector 22. In this case, the size and power consumption of the pulse wave sensor 2 become larger than this embodiment.

In the above embodiment, the acrylic transparent plate 6 is provided on the detection face 23a of the holder 23 to improve adhesion to the wrist 10. Even when the detection face 23a is projected from the outer casing 5 as shown in FIG. 5, adhesion can be improved.

In the above embodiment, the pulse rate data is transmitted to the display or the device for computing the amount of motion load. When not only a pulse rate but also pulse wave data (waveform itself) are transmitted, the pulse rate detector 1 of the present invention can be coupled to devices making use of bio signals.

As described above, according to the present invention, since a pulse wave sensor is constituted such that light output from a light emitting diode and reflected from the artery of the wrist of a subject is detected by at least three photodetectors disposed around the light emitting diode and not linearly to detect a pulse wave, even when the attachment position of the sensor is dislocated, the pulse wave can be detected accurately. Using this sensor, a pulse rate detector which is easily attached and has a stable output can be constructed.

What is claimed is:

1. A pulse wave sensor for detecting a pulse wave by detecting light output from a light emitting diode and reflected from the artery of a wrist of a subject, the sensor comprising at least three photodetectors disposed around the light emitting diode.

2. The pulse wave sensor of claim 1, wherein a near infrared LED is used as the light emitting diode.

3. The pulse wave sensor of claim 1, wherein the photodetectors are disposed at an equal distance from the light emitting diode.

4. The pulse sensor of claim 1, wherein cavities are formed in a contact face between a holder for holding the light emitting diode and the photodetectors and the wrist, the light emitting face of the light emitting diode and the light receiving faces of the photodetectors are disposed at respective predetermined distances from the contact face, and the sectional forms of the cavities are tapered such that their widths increase toward the contact face.

5. The pulse wave sensor of claim 1, wherein a transparent plate-like member is provided on a portion including at least the light emitting face and the light receiving faces of the contact face.

6. A pulse rate detector comprising the pulse wave sensor of claim 1 and means of computing the pulse rate of a subject based on the output of the pulse wave sensor.

7. The pulse rate detector of claim 6 which comprises a transmitter for transmitting the measured pulse rate data.
